(19) United States
(12) Patent Application Publication
(10) Pub. No.: US 2023/0125282 A1
(43) Pub. Date: Apr. 27, 2023

(54) Sensor Device

(71) Applicant: Cognito Health Inc., Taipei City (TW)

(72) Inventors: Li-Chin Ho, Taipei City (TW); Tsai-Yu Lin, Taipei City (TW)

(73) Assignee: Cognito Health Inc., Taipei City (TW)

(21) Appl. No.: 17/507,799

(22) Filed: Oct. 21, 2021

(51) Int. Cl. H05K 1/18

(52) U.S. Cl. CPC H05K 1/189; H05K 1/181; H05K 2201/10151; A61B 5/6801

(57) ABSTRACT

A sensor device adapted to suit curved surfaces includes a flexible substrate, a plurality of sensing units and a plurality of connecting lines. The flexible substrate includes a plurality of connecting sections, wherein each of the plurality of connecting sections comprises at least one closed hollow region. A plurality of sensing units, disposed on the flexible substrate and arranged in an array; and a plurality of connecting lines, disposed on the flexible substrate. Each of the plurality of connecting lines is connected to two adjacent ones of the plurality of sensing units, and the plurality of connecting sections are respectively overlapped with the plurality of connecting lines.

### BACKGROUND OF THE INVENTION

### 1. Field of the Invention

The present invention relates to a sensor device, and more particularly, to a sensor device capable of being adapted to a variety of curved surface, difficult to break, and having high ductility.

### 2. Description of the Prior Art

Medical sensor devices for medical diagnosis and treatment are more and more important. Existing medical sensor devices mostly are rigid, and may not be laid on the irregular curved surface and change shape with the curved surface, or may not be directly in contact with the body part to be measured or treated and change shape with the body part. These limitations affect accuracy of measurement and effectiveness of the treatment. Further, the conventional medical sensors are probably constituted by an integral-surfaced substrate without holes, and therefore have poor ventilation and low ductility, such that sensitivity or accuracy of sensing is susceptible to wrinkle or deformation.

### SUMMARY OF THE INVENTION

It is therefore an objective of the present invention to provide a sensor device capable of being adapted to a variety of curved surface, difficult to break, and having high ductility.

The present invention discloses a sensor device adapted to suit curved surfaces includes a flexible substrate, a plurality of sensing units, and a plurality of connecting lines. A flexible substrate, including a plurality of connecting sections, wherein each of the plurality of connecting sections comprises at least one closed hollow region; a plurality of sensing units, disposed on the flexible substrate and arranged in an array; and a plurality of connecting lines, disposed on the flexible substrate, wherein each of the plurality of connecting lines is connected to two adjacent ones of the plurality of sensing units, and the plurality of connecting sections are respectively overlapped with the plurality of connecting lines.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1A is a partial schematic diagram of a sensor device when not being stretched according to an embodiment of the present invention.

FIG. 1B is a partial schematic diagram of the sensor device of FIG. 1A when being stretched according to an embodiment of the present invention.

FIG. 2 to FIG. 8 are partial schematic diagrams of flexible substrates 200 to 800 according to embodiments of the present invention.

FIG. 9 is a partial schematic diagram of a flexible substrate according to an embodiment of the present invention.

### DETAILED DESCRIPTION

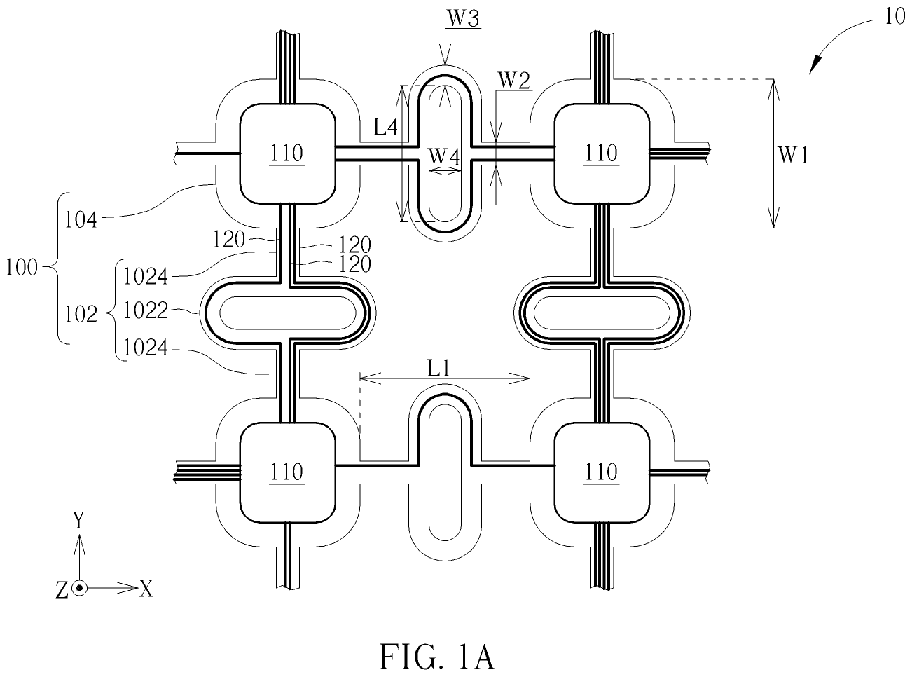

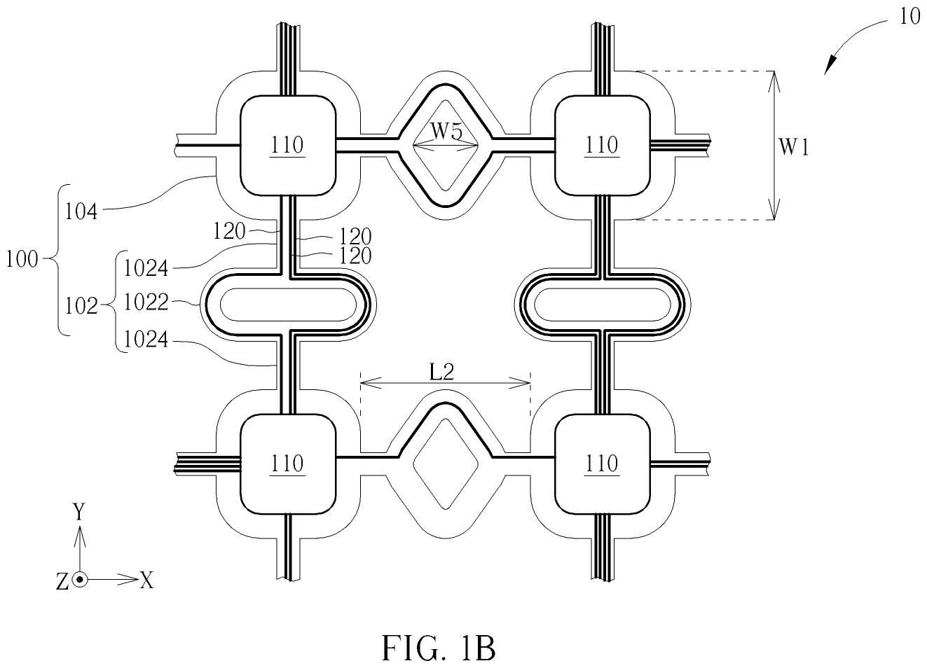

Please refer to FIG. 1A and FIG. 1B. FIG. 1A is a partial schematic diagram of a sensor device 10 when not being stretched according to an embodiment of the present invention. FIG. 1B is a partial schematic diagram of the sensor device 10 of FIG. 1A when being stretched along a direction X according to an embodiment of the present invention. The sensing device 10 includes a flexible substrate 100, sensing units 110 and connecting lines 120. The flexible substrate 100 includes connecting sections 102 and element sections 104, and each connecting section 102 is connected between two adjacent element sections 104. As shown in FIG. 1A, each connecting section 102 includes a closed hollow region 1022 and strip regions 1024. The closed hollow region 1022 is connected between the adjacent two strip regions 1024, and thus are located between two opposing ends of the connecting section 102. The sensing units 110 are respectively disposed on the element sections 104 of the flexible substrate 100 and thus overlapped with the element sections 104, and arranged in an array. The connecting lines 120 are respectively disposed on the connecting sections 102 of the flexible substrate 100 and thus overlapped with the connecting sections 102. Each connecting line 120 is connected between two adjacent sensing units 110, to transmit signals between the two adjacent sensing units 110.

In short, compared to a substrate without holes, the sensor device 10 forms a hollow structure by connecting the connecting sections 102 between two adjacent element sections 104, so the sensor device 10 may enhance air permeability and ductility of the sensor device 10, and may be adapted to a variety of curved surfaces, and may prevent wrinkle or deformation from affecting the sensitivity or accuracy of the sensor device 10. In addition, the closed hollow regions 1022 of the sensor device 10 may further enhance ductility of the sensor device 10, and increase reliability and be difficult to break, thereby preventing the sensing unit 110 from rotating when the sensor device 10 is stretched.

Specifically, as shown in FIG. 1B, when the sensing device 10 is stretched along the direction X, the sensing units 110 and the connecting lines 120 may extend with the flexible substrate 100. Further, shapes of the closed hollow regions 1022 of the connecting sections 102 may be appropriately changed. For example, lower and upper closed hollow regions 1022 are changed from pill shapes shown in FIG. 1A into diamond shapes shown in FIG. 1B. As a result, the effective length of the connecting section 102 may be further increased, to improve the ductility. Accordingly, the sensor device 10 may be transformed from a length L1 shown in FIG. 1A to a length L2 shown in FIG. 1B. Thus, a unit length extension amount of the element section 104 of the flexible substrate 100 may be different from a unit length extension amount of the closed hollow region 1022. That is, when the sensor device 10 is stretched along a direction (e.g. the direction X), the length change of the unit length of the element section 104 before (or after) stretched in this direction is smaller than the length change of unit length of the closed hollow region 1022 before (or after) stretched in this direction.

In some embodiments, when the sensor device 10 is locally pressed along the direction Z, the sensor device 10 is able to and may be stretched in a plurality of directions (such as direction X or direction Y) to dispersed the tension. As a result, the sensor device 10 may be adapted to various curved surfaces, and prevent wrinkle or deformation from affecting the sensitivity or accuracy of the sensor device 10. Accordingly, the sensor device 10 may be laid on soft materials such as mattresses, cushions, or insoles, to detect temperature, pressure, or movement, humidity, or emission material type of a non-planar object (such as a human body). In this case, the sensing unit 110 may correspondingly be a temperature detector, a pressure detector, an accelerometer, a gyroscope, a humidity detector, a fluid detector or a chemical substance detector, but is not limited to these. Further, if the sensing sensor device 10 is stretched within the elastic limit, deformation of the sensor device 10 is reversible. That is, after the external force is removed, the sensor device 10 restores.

In order to optimize the ductility of the sensor device 10, geometry ratio of the flexible substrate 100 may be adjusted depending on different design considerations. For example, table 1 to table 3 respectively lists the relationship between geometry ratio and extension amount of the flexible substrate 100. As shown in FIG. 1A and FIG. 1B, the closed hollow region 1022 may be defined according to a hollow outline (such as a pill shape or a diamond shape). In the table 1 to table 3, W2 is a width (or referred to as a second width) of the strip region 1024 of the flexible substrate 100 when not being stretched, W3 is a width (or referred to as a third width) of the closed hollow region 1022 of the flexible substrate 100 when not being stretched, W4 is a width (or referred to as a fourth width) of the hollow outline of the closed hollow region 1022 when not being stretched, L4 is a length (or referred to as a first length) of the hollow outline of the closed hollow region 1022 when not being stretched, dW is a difference (namely, the extension amount of the closed hollow region 1022) between a width W4 and a width W5 of the hollow outline of the closed hollow region 1022 when not being stretched and being stretched, respectively. In table 1, W3:W4=1:1; in table 2, W3:W4=1:2; in table 3, W3:W4=1:3. Table 1 to Table 3 shows that when the ratio of W3:W2 is less, the extension amount dW of the closed hollow region 1022 is less; that is, the percent elongation of the flexible substrate 100 is relatively less. Alternatively, when the ratio of W4:L4 is less, the extension amount dW of the closed hollow region 1022 is greater; similarly, when the ratio of W3:W4 is less, the extension amount dW of the closed hollow region 1022 is greater. Further, the extended amount dW of the closed hollow region 1022 is related to the width W2 of the stripe region 1024, the width W3 and the length L4 of the closed hollow region 1022, the width W4 of the hollow outline of the closed hollow region 1022.

TABLE 1

| | W4:L4 = | W4:L4 = | W4:L4 = | W4:L4 = |
|---|---|---|---|---|
| | 1:2 | 1:3 | 1:4 | 1:5 |
| W3:W2 = | L4:dW = | L4:dW = | L4:dW = | L4:dW = |
| 1:1 | 1:0.14 | 1:0.23 | 1:0.32 | 1:0.4 |
| W3:W2 = | L4:dW = | L4:dW = | L4:dW = | L4:dW = |
| 1:2 | 1:0.07 | 1:0.09 | 1:0.17 | 1:0.28 |
| W3:W2 = | | L4:dW = | L4:dW = | L4:dW = |
| 1:3 | | 1:0.04 | 1:0.1 | 1:0.17 |
| W3:W2 = | | | L4:dW = | L4:dW = |
| 1:4 | | | 1:0.03 | 1:0.11 |
| W3:W2 = | | | | L4:dW = |
| 1:5 | | | | 1:0.05 |

TABLE 2

| | W4:L4 = | W4:L4 = | W4:L4 = | W4:L4 = | W4:L4 = |
|---|---|---|---|---|---|
| | 1:1 | 1:2 | 1:3 | 1:4 | 1:5 |
| W3:W2 = | L4:dW = | L4:dW = | L4:dW = | L4:dW = | L4:dW = |
| 1:1 | 1:0.14 | 1:0.35 | 1:0.38 | 1:0.57 | 1:0.62 |
| W3:W2 = | | L4:dW = | L4:dW = | L4:dW = | L4:dW = |
| 1:2 | | 1:0.14 | 1:0.23 | 1:0.39 | 1:0.54 |
| W3:W2 = | | L4:dW = | L4:dW = | L4:dW = | L4:dW = |
| 1:3 | | 1:0.07 | 1:0.19 | 1:0.32 | 1:0.37 |
| W3:W2 = | | L4:dW = | L4:dW = | L4:dW = | L4:dW = |
| 1:4 | | 1:0.03 | 1:0.09 | 1:0.25 | 1:0.34 |
| W3:W2 = | | | L4:dW = | L4:dW = | L4:dW = |
| 1:5 | | | 1:0.04 | 1:0.14 | 1:0.25 |

TABLE 3

| | W4:L4 = | W4:L4 = | W4:L4 = | W4:L4 = | W4:L4 = |
|---|---|---|---|---|---|
| | 1:1 | 1:2 | 1:3 | 1:4 | 1:5 |
| W3:W2 = | L4:dW = | L4:dW = | L4:dW = | L4:dW = | L4:dW = |
| 1:1 | 1:0.2 | 1:0.4 | 1:0.5 | 1:0.62 | 1:0.7 |
| W3:W2 = | L4:dW = | L4:dW = | L4:dW = | L4:dW = | L4:dW = |
| 1:2 | 1:0.5 | 1:0.25 | 1:0.43 | 1:0.55 | 1:0.66 |
| W3:W2 = | | L4:dW = | L4:dW = | L4:dW = | L4:dW = |
| 1:3 | | 1:0.2 | 1:0.36 | 1:0.47 | 1:0.58 |
| W3:W2 = | | L4:dW = | L4:dW = | L4:dW = | L4:dW = |
| 1:4 | | 1:0.1 | 1:0.23 | 1:0.37 | 1:0.48 |
| W3:W2 = | | L4:dW = | L4:dW = | L4:dW = | L4:dW = |
| 1:5 | | 1:0.05 | 1:0.13 | 1:0.27 | 1:0.42 |

In order to enhance structural strength of the sensor device 10, geometry ratio of the flexible substrate 100 may be adjusted depending on different design considerations. In some embodiments, a width W2 of the connecting section 102 of the flexible substrate 100 may be less than a width W1 (or referred to as a first width) of the element section 104, but is not Limited to this. In some embodiments, in order to improve reliability, the connecting section 102 may be symmetric with respect to the direction X (or referred to as a first direction) and the direction Y (or referred to as a second direction). In some embodiments, in order to improve reliability, the closed hollow region 1022 may have a hollow structure defined according to a hollow outline, so that the effective width of the connecting section 102 at the closed hollow region 1022 is essentially increased. That is, by having the symmetric connecting section 102 and improving the effective width of the connecting section 102, the tension may be uniformly dispersed and the structural strength is enhanced, to prevent the connecting section 102 from breaking or peeling, thereby improving reliability. Further, since the connecting section 102 is symmetric, it prevents the sensing unit 110 from rotating when the sensor device 10 is stretched, and avoids friction between the sensing unit 110 and the flexible substrate 100 or between different layers of the flexible substrate 100, thereby ensuring lifecycle of the sensing unit 110.

In some embodiments, the flexible substrate 100 may be made of a polymer, such as polyimide (PI), polyamide (PA), polymerized siloxanes (polysiloxanes), polyurethane (PU) or polyester. In some embodiments, the flexible substrate 100 may have a single-layer or multi-layer structure. Similarly, the sensing unit 110 or the connecting line 120 may also have a single-layer or multi-layer structure. In some embodiments, the connecting line 120 may be made of a highly conductive metal (for example, copper or gold). In some embodiments, the sensor device 10 may be a flexible printed circuit board (FPC) or manufactured by a manufacturing method of the flexible printed circuit board.

In some embodiments, in order to enhance the reliability, the connecting line 120 may be disposed in the connecting section 102 of the flexible substrate 100 and partially or completely covered by the connecting section 102, to prevent the connecting section 102 and the connecting lines 120 from breaking or peeling, thereby improving reliability. In some embodiments, the sensor device 10 may further include a reinforcing layer for a covering the flexible substrate 100, to increase the structural strength.

As shown in FIG. 1A, distribution of the connecting lines 120 may be adjusted depending on various design considerations. In some embodiments, the closed hollow region 1022 may be partially or completely surrounded by the connecting lines 120. For example, as shown in FIG. 1A, the upper, left, and right closed hollow regions 1022 are completely surrounded by the connecting lines 120, and the lower closed hollow region 1022 is partially surrounded by a connecting lines 120. In some embodiments, distribution of the connecting lines 120 in the closed hollow region 1022 may be symmetric or asymmetric. For example, as shown in FIG. 1A, distribution of the connecting lines 120 in the upper and right closed hollow regions 1022 is symmetric, and distribution of the connecting lines 120 in the lower and left closed hollow regions 1022 is asymmetrical.

It should be noted that the sensor device 10 is an embodiment of the present invention, and those skilled in the art may make various alterations and modifications accordingly. For example, in some embodiments, the sensor device 10 may further include an adhesive layer, for adhering the flexible substrate 100 over a non-planar object. In some embodiments, the sensor device 10 may further include a protective layer for covering the flexible substrate 100, to provide isolation against liquids, moisture, dust and other substances, or to improve the comfort of the surface. In some embodiments, the protective layer may be formed by coating treatment. In some embodiments, the sensing unit 110 may include a processing circuit, a storage module, a power supply module, and a communication interface. The processing circuit may be a microprocessor or an application-specific integrated circuit (ASIC), but is not limited thereto. The storage module may be a subscriber identity module (SIM), a read-only memory (ROM), a flash memory or a random access memory (RAM), but not limited to this. The power module may be a battery or a solar panel. The communication interface may be a transceiver, for example, a wireless transceiver for transmitting and receiving signals (e.g. messages or packets, etc.), but is not limited thereto.

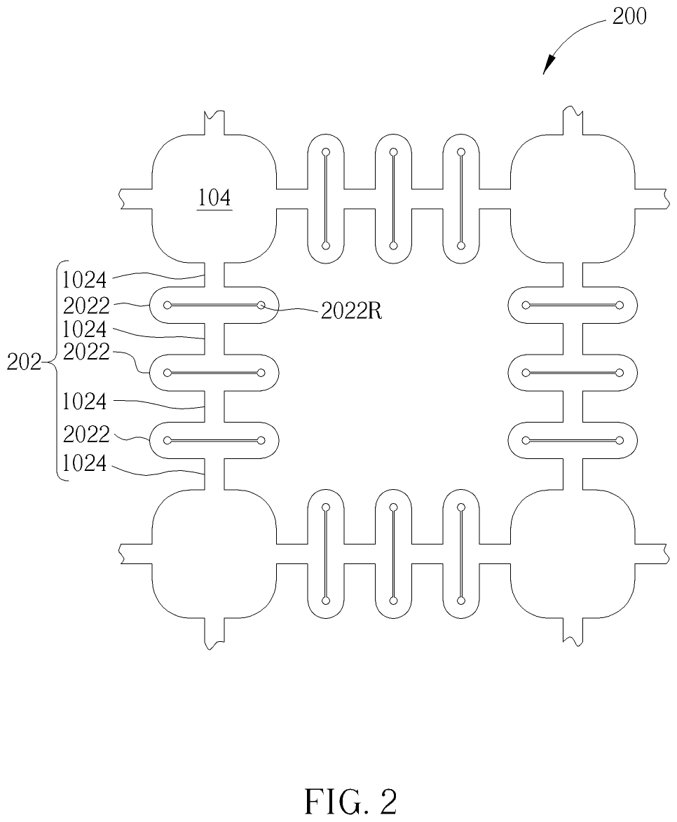

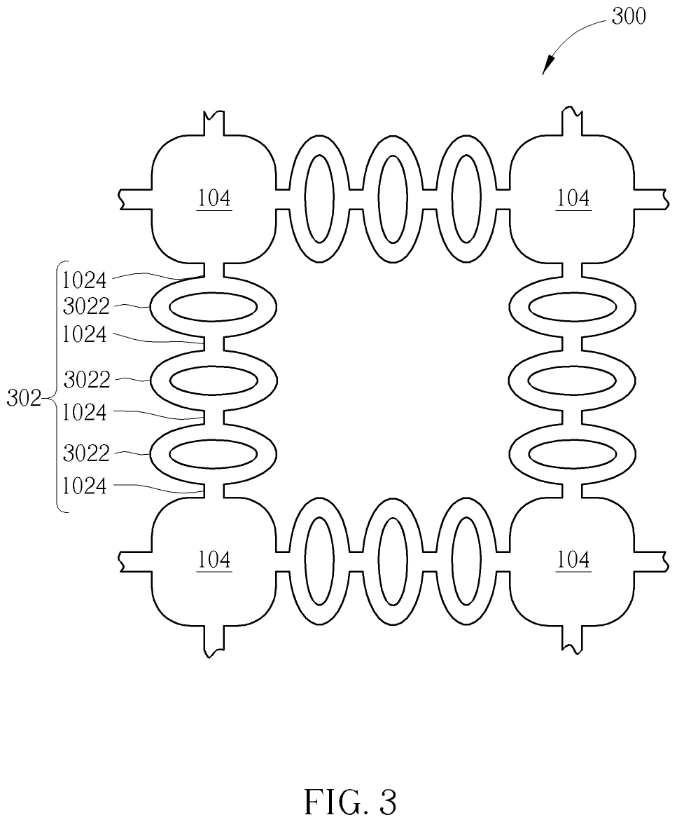

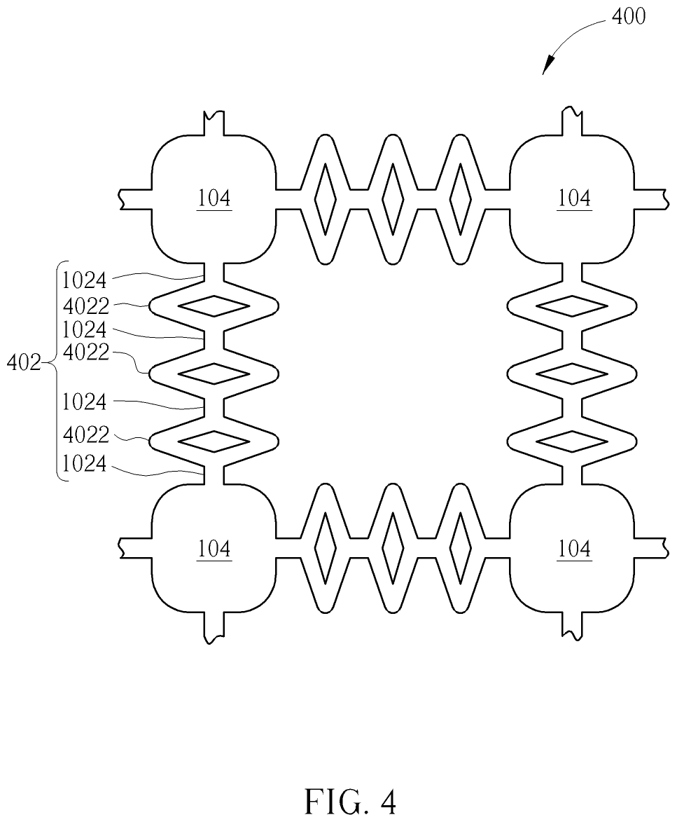

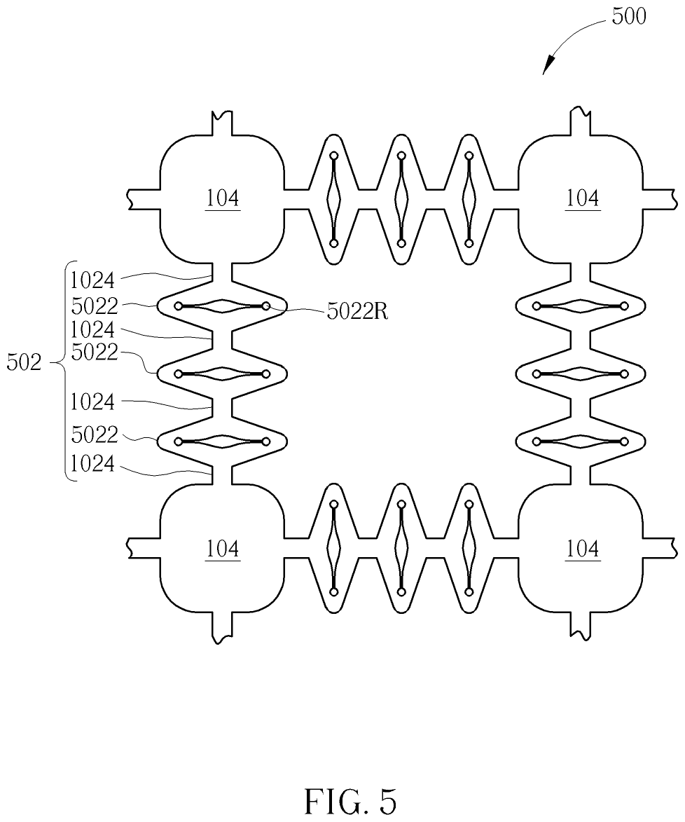

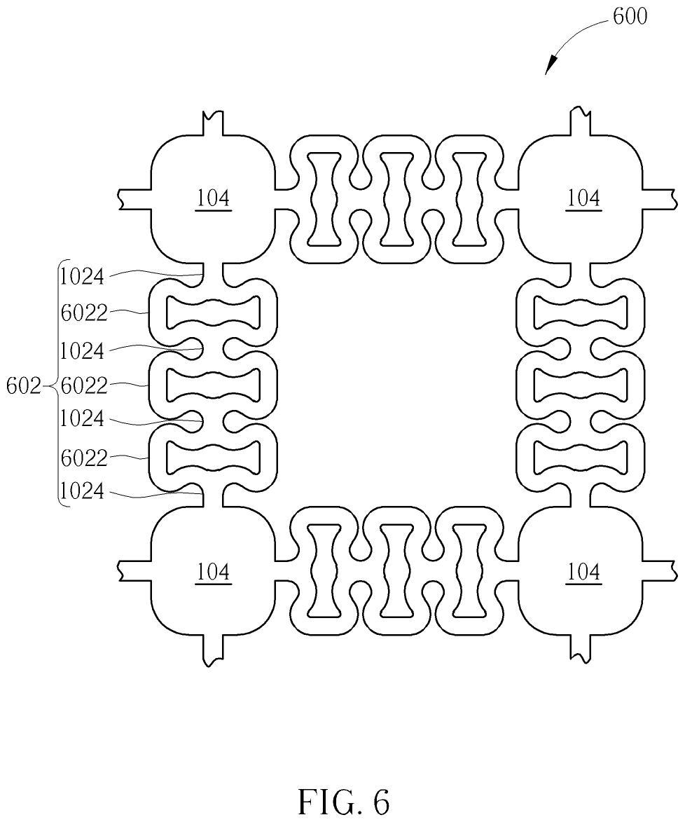

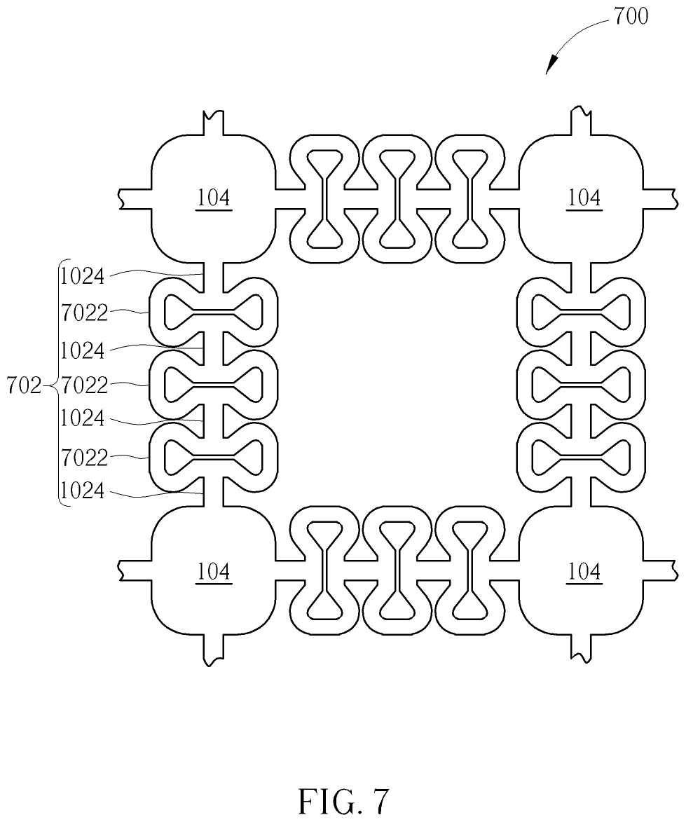

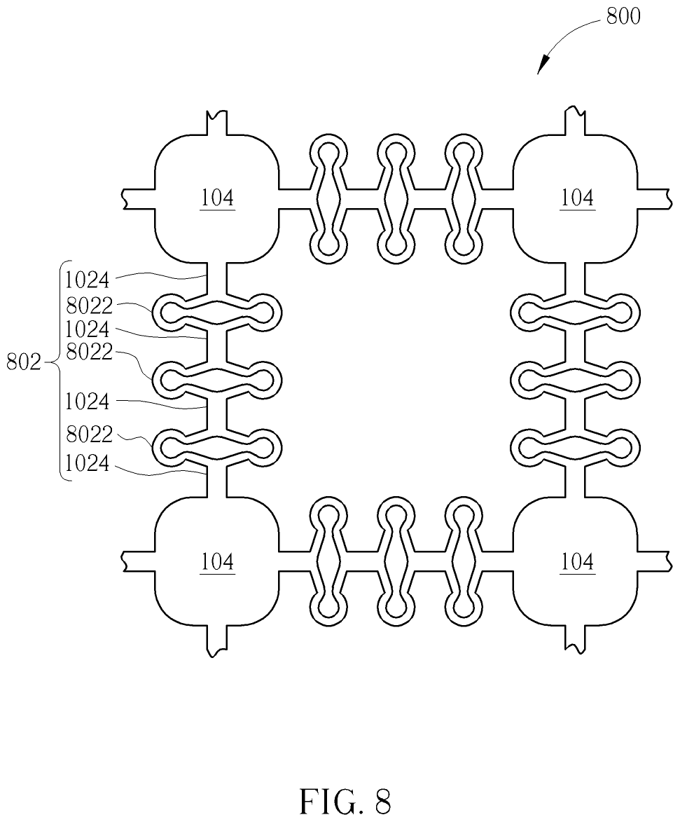

The outline of the closed hollow region 1022 may be adjusted according to different design considerations. In some embodiments, the outline of the closed hollow region 1022 may be a shape of a circle, an oval, a diamond, a polygon, a dumbbell, a pill, a funnel, or other combination of patterns, but not limited to this. For example, please refer to FIG. 2 to FIG. 8. FIG. 2 to FIG. 8 are partial schematic diagrams of flexible substrates 200 to 800 according to embodiments of the present invention. Structures of the flexible substrates 200 to 800 are similar to the flexible substrate 100, and thus the same elements are denoted by the same identifiers. The flexible substrates 200 to 800 respectively include connecting sections 202 to 802 and the element section 104. The connecting sections 202-802 respectively include closed hollow regions 2022-8022 and strip regions 1024. The closed hollow region 2022-8022 respectively have different shapes of the hollow outlines. Further, the closed hollow region 2022, 5022, respectively, include stop hole 2022R, 5022R, to further increase the structural strength, to prevent the closed hollow regions 2022, 5022 from tearing, thereby improving reliability. In addition the a number of the closed hollow regions 1022 between two adjacent element sections 104 may be adjusted depending on different design considerations. In some embodiments, a plurality of closed hollow regions 1022 may be connected in series through a plurality of strip regions 1024, to further enhance the ductility of the sensor device 10. For example, as shown in FIG. 2 to FIG. 8, 3 closed hollow regions 2022-8022 are respectively disposed between two adjacent element sections 104, and connected in series via 4 strip regions 1024.

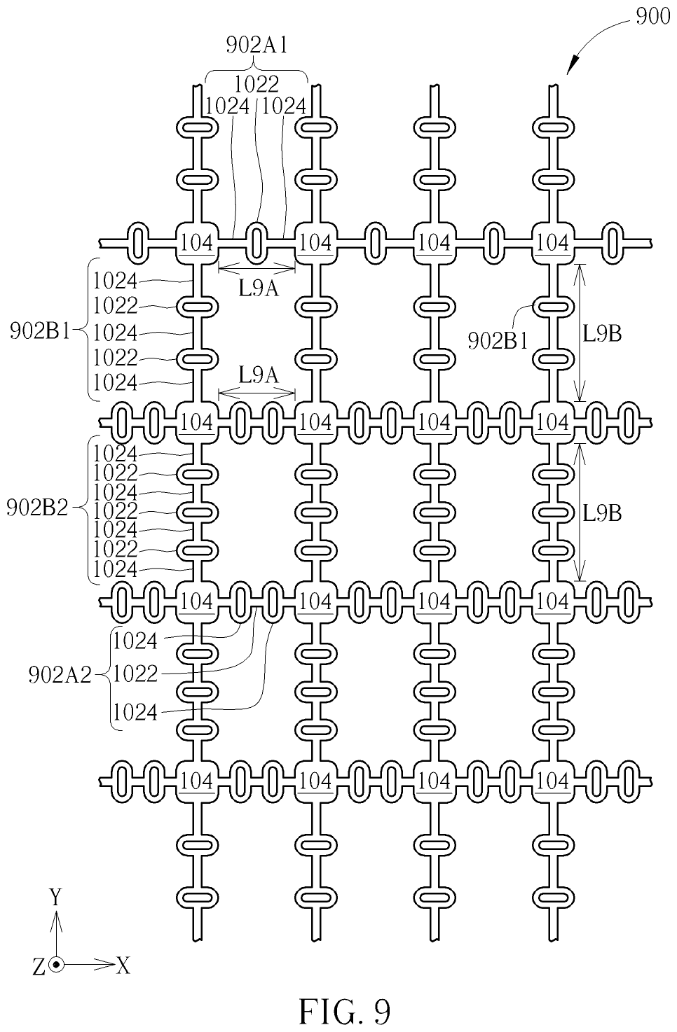

Lengths of different connecting sections 102 or the number of closed hollow regions 1022 may be adjusted according to different design considerations. For example, please refer to FIG. 9. FIG. 9 is a partial schematic diagram of a flexible substrate 900 according to an embodiment of the present invention. Structure of the flexible substrate 900 is similar to that of the flexible substrate 100, and thus the same elements are denoted by the same identifiers. Difference is that, connecting sections of the flexible substrate 900 may be divided into connecting sections 902A1, 902A2, 902B1, 902B2. The connecting sections 902A1, 902A2 are parallel to the direction X, and the connecting sections 902B1, 902B2 are parallel to the direction Y. In addition, a length L9A (or called a second length) of the connecting sections 902A1, 902A2 is different from a length L9B (or referred to as a third length) of the connecting sections 902B1, 902B2. In some embodiments, the number of closed hollow regions 1022 in the connecting sections 902A1, 902A2, 902B1, and 902B2 may be different. As shown in FIG. 9, the connecting section 902A1 includes 1 closed hollow region 1022, the connecting section 902A2 includes 2 closed hollow regions 1022, the connecting section 902B1 includes 2 closed hollow regions 1022, and the connecting section 902B2 includes 3 closed hollow regions 1022.

In summary, compared with a substrate without holes, the sensor device 10 of the present invention forms a hollow structure by connecting the connecting section 102 between two adjacent element sections 104. Thus, the sensor device 10 may improve the air permeability of the sensor device 10, and may have high ductility to be adapted to various curved surfaces, and may prevent wrinkle or deformation from affecting the sensitivity or accuracy of the sensor device 10. Further, the closed hollow region 1022 of the present invention may further enhance the ductility of the sensor device 10, and increase reliability and be difficult to break, and may prevent the sensing unit 110 from rotating when the sensor device 10 is stretched.

Those skilled in the art will readily observe that numerous modifications and alterations of the device and method may be made while retaining the teachings of the invention. Accordingly, the above disclosure should be construed as limited only by the metes and bounds of the appended claims.

1. A sensor device, adapted to suit curved surfaces, comprising:
a flexible substrate, comprising a plurality of connecting sections, wherein each of the plurality of connecting sections comprises at least one closed hollow region;
a plurality of sensing units, disposed on the flexible substrate and arranged in an array; and
a plurality of connecting lines, disposed on the flexible substrate, wherein each of the plurality of connecting lines is connected to two adjacent ones of the plurality of sensing units, the plurality of connecting sections are respectively overlapped with the plurality of connecting lines, and the plurality of connecting sections comprises a plurality of first connecting sections and a plurality of second connecting sections, and a second length of the plurality of first connecting sections is different from a third length of the plurality of second connecting sections.

2. The sensor device of claim 1, wherein the flexible substrate further comprises a plurality of element sections, the plurality of element sections are overlapped with the plurality of sensing units, and when the sensor device is stretched, at least one unit length extension amount of the plurality of element sections is different from the unit length extension amount of the closed hollow region.

3. The sensor device of claim 2, wherein each of a first width of the plurality of element sections is greater than each of a second width of the plurality of connecting sections.

4. The sensor device of claim 1, wherein the at least one closed hollow region has a third width, and each of the at least one closed hollow region is defined according to a hollow outline, and the hollow outline has a fourth width and a first length, and an extension amount of each of the at least one closed hollow region is related to the second width, the third width, the fourth width or the first length.

5. The sensor device of claim 1, wherein a hollow outline of each of the at least one closed hollow region is a shape of a circle, an oval, a diamond, a polygon, a pill or a funnel.

6. The sensor device of claim 1, wherein the plurality of connecting sections are symmetric with respect to a first direction or a second direction, and the first direction is perpendicular to the second direction.

7. The sensor device of claim 1, wherein a number of the at least one closed hollow region of one of the plurality of connecting sections is different from another number of the at least one closed hollow region of another of the plurality of connecting sections.

8. (canceled)

9. (canceled)

10. The sensor device of claim 1, wherein the at least one closed hollow region is partially or completely surrounded by the plurality of connecting lines.

11. The sensor device of claim 1, wherein a hollow outline of each of the at least one closed hollow region is a shape of a dumbbell.

12. The sensor device of claim 1, wherein at least one first closed hollow region of the at least one closed hollow region is located between two adjacent ones of the plurality of sensing units, at least one second closed hollow region of the at least one closed hollow region is located between another two adjacent ones of the plurality of sensing units, and a number of the at least one first closed hollow region is different from a number of the at least one second closed hollow region.

13. The sensor device of claim 1, wherein a hollow outline of one of the at least one closed hollow region comprises:
a first segment, having a shape of a circle or a triangle;
a second segment, having a shape of a circle or a triangle; and
a third segment, having a shape of a bar and disposed between the first segment and the second segment, wherein a third width of the third segment is smaller than a first width of the first segment and a second width of the second segment, and a third length of the third segment is larger than a first length of the first segment and a second length of the second segment.

14. The sensor device of claim 1, wherein a hollow outline of one of the at least one closed hollow region comprises:
a first segment, having a shape of a circle;
a second segment, having a shape of a circle; and
a third segment, having a shape of a diamond and disposed between the first segment and the second segment, wherein a third width of the third segment is larger than a first width of the first segment and a second width of the second segment, and a third length of the third segment is larger than a first length of the first segment and a second length of the second segment.
